(19) United States
(12) Patent Application Publication
(10) Pub. No.: US 2024/0182430 A1
(43) Pub. Date: Jun. 6, 2024

(54) SYNTHESIS OF SUBSTITUTED PYRAZINES

(71) Applicant: MediBeacon Inc., St. Louis, MO (US)

(72) Inventors: Thomas E. Rogers, Ballwin, MO (US); Eric Buck, Chicago, IL (US)

(21) Appl. No.: 18/520,826

(22) Filed: Nov. 28, 2023

Related U.S. Application Data
(60) Provisional application No. 63/385,098, filed on Nov. 28, 2022.

(51) Int. Cl. C07D 241/28

(52) U.S. Cl. CPC C07D 241/28

(57) ABSTRACT

Described herein are methods to form substituted pyrazines. Also described herein are methods to purify substituted pyrazines.

### CROSS-REFERENCE TO RELATED APPLICATION

This application claims priority to U.S. Provisional Application No. 63/385,098, filed Nov. 28, 2022, the contents of which are hereby incorporated by reference herein.

### BACKGROUND OF THE DISCLOSURE

The field of the disclosure relates generally to chemical synthesis and purification of substituted pyrazines.

More particularly, the disclosure relates to novel chemical synthesis methods including catalytic hydrogenation to form substituted pyrazines that are used in the preparation of higher value products, such as imaging agents, as well as novel precipitation-based purification methods to purify substituted pyrazines.

Methods for the preparation of substituted pyrazines are known in the art. Problematically, known methods provide low yields and significant impurities. For instance, catalytic hydrogenation can result in an over-reduction of the desired compound. These low yields and impurities limit the use of catalytic hydrogenation to prepare substituted pyrazines.

A need therefore exists for improved processes for preparing and purifying substituted pyrazines that improve yields.

### BRIEF DESCRIPTION

In some aspects, described herein is a method of preparing a compound of Formula II or a salt thereof,

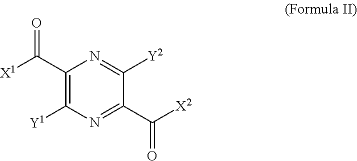

the method comprising:
forming a mixture comprising
   a compound of Formula I or a salt thereof,

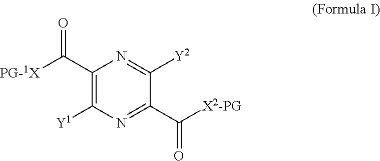

wherein PG, X¹, X², Y¹, and Y² are as defined anywhere in this disclosure;
   an organic solvent;
   a catalyst; and
   a hydrogenation agent; and
reacting the mixture.

In some aspects, described herein is a method of purifying a compound of Formula II or a salt thereof,

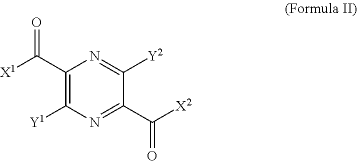

the method comprising:
precipitating the compound of Formula II or a salt thereof with a first solvent and an anti-solvent; and
optionally removing the first solvent from the compound of Formula II or a salt thereof;
   wherein X¹, X², Y¹, and Y² are as defined anywhere in this disclosure.

### BRIEF DESCRIPTION OF THE DRAWINGS

FIG. 1 is an exemplary method flow chart in accordance with the present disclosure.

FIG. 2 is a first representative reaction in accordance with the present disclosure.

FIG. 3 is a second representative reaction in accordance with the present disclosure.

FIG. 4 depicts high performance liquid chromatography (HPLC) chromatograms obtained after partial completion of a reaction in accordance with the present disclosure. The top and bottom chromatograms differ only in scale.

FIG. 5 depicts high performance liquid chromatography (HPLC) chromatograms obtained after completion of a reaction in accordance with the present disclosure. The top and bottom chromatograms differ only in scale.

FIG. 6 depicts high performance liquid chromatography (HPLC) chromatograms obtained at an absorption wavelength of 264 nm after completion of an aqueous reaction not in accordance with the present disclosure. The top and bottom chromatograms differ only in scale.

FIG. 7 depicts high performance liquid chromatography (HPLC) chromatograms obtained at an absorption wavelength of 215 nm after completion of an aqueous reaction not in accordance with the present disclosure. The top and bottom chromatograms differ only in scale.

FIG. 8 is an exemplary method flow chart in accordance with the present disclosure.

### DETAILED DESCRIPTION

The present disclosure is generally directed to improved methods for the preparation of a compound of Formula II and/or salts thereof from a compound of Formula I or a salt thereof. Formula II and salts thereof is an imaging compound useful as a fluorescent dye, for instance and without limitation, for evaluating renal function and for imaging vasculature, such as eye vasculature.

It was surprisingly discovered herein that catalytic hydrogenation of protected substituted pyrazines could be achieved with the use of an organic solvent. In a particular embodiment, the use of ethyl acetate as a co-solvent with ethanol instead of water increases yields from 35-70% to over 90%.

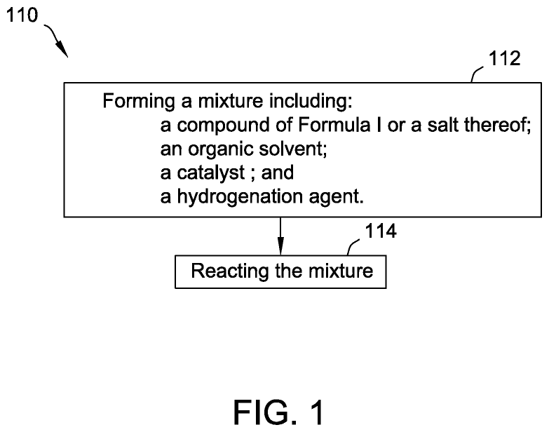

FIG. 1 is an exemplary method flow chart 110. In this exemplary embodiment, method flow chart 110 depicts the essential method steps of the method embodiments described herein and is not intended to limit the method embodiments. Method step 112 includes forming a mixture including: a compound of Formula I or a salt thereof; an organic solvent; a catalyst; and a hydrogenation agent. Method step 114 includes reacting the mixture.

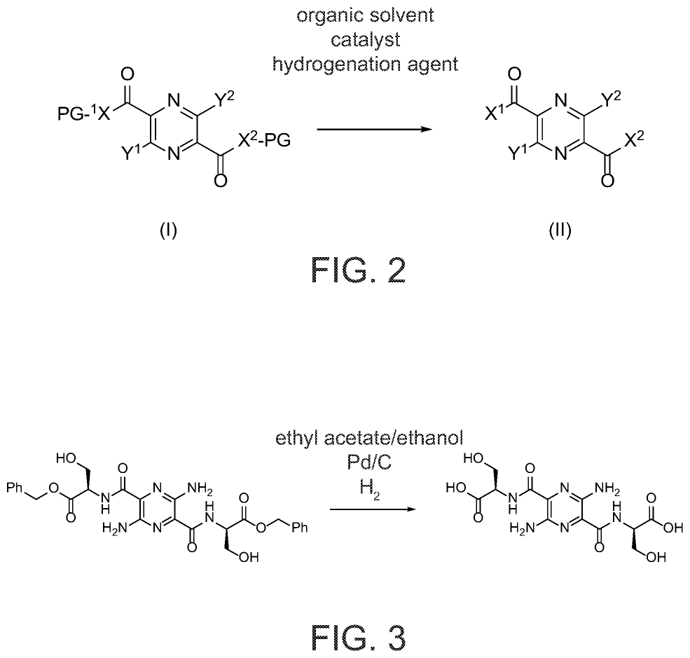

In one aspect, a compound of Formula II or a salt thereof is prepared according to a method represented by FIG. 2. PG, X¹, X², Y¹, and Y² are as defined anywhere in this disclosure. X¹ is shown as ¹X in Formula I to improve visual clarity.

This aspect includes forming a mixture including a compound of Formula I or a salt thereof, an organic solvent, a catalyst, and a hydrogenation agent, and then reacting the mixture.

In some embodiments, each of Y¹ and Y² is independently selected from hydrogen, halogen, amine, nitroso, nitro, amide, ester, and carboxyl; and

In some embodiments, each of X¹ and X² is independently one or more natural or unnatural α-amino acids or a polypeptide chain that includes one or more natural or unnatural α-amino acids linked together by peptide bonds. The polypeptide chain (AA) may be a homopolypeptide chain or a heteropolypeptide chain, and may be any appropriate length. For instance, in some embodiments, the polypeptide chain may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 α-amino acid(s), 1 to 90 α-amino acid(s), 1 to 80 α-amino acid(s), 1 to 70 α-amino acid(s), 1 to 60 α-amino acid(s), 1 to 50 α-amino acid(s), 1 to 40 α-amino acid(s), 1 to 30 α-amino acid(s), 1 to 20 α-amino acid(s), or even 1 to 10 α-amino acid(s). In some embodiments, the α-amino acids of the polypeptide chain (AA) are selected from aspartic acid, asparagine, arginine, histidine, lysine, glutamic acid, glutamine, serine, and homoserine. In some embodiments, the α-amino acids of the polypeptide chain (AA) are selected from aspartic acid, glutamic acid, serine, and homoserine. In some embodiments, the polypeptide chain (AA) refers to a single amino acid (e.g., aspartic acid or serine). In some embodiments the amino acid is D-serine.

In some embodiments, each of X¹ and X² is independently N(R¹)(R²).

In these embodiments, each R¹ is independently —(CH₂)ₐ(CH₂CH₂O)_{b}(CH₂)_{c}NR¹⁰CONR¹¹(CH₂)_{d}(CH₂CH₂O)ₑR²⁰, —(CH₂)ₐ(CH₂CH₂O)_{b}(CH2)_{c}NR¹²CSNR¹³(CH₂)_{d}(CH₂CH₂O)ₑR²¹, —(CH₂)ₐ(CH₂CH₂O)_{b}(CH₂)_{c}CONR¹⁴(CH₂)_{d}(CH₂CH₂O)ₑR²², —(CH₂)ₐ(CH₂CH₂O)_{b}(CH₂)_{c}NR¹⁵SO₂(CH₂)_{d}(CH₂CH₂O)ₑR²³, —CH₂)ₐ(CH₂CH₂O)_{b}(CH₂)_{c}SO₂NR¹⁶(CH₂)_{d}(CH₂CH₂O)ₑR²⁴, —(CH2)ₐ(CH₂CH₂O)_{b}(CH₂)_{c}NR¹⁷CO(CH₂)_{d}(CH₂CH₂O)ₑR²⁵, —(CH2)ₐ(CH₂CH₂O)_{b}(CH₂)_{c}NR¹⁸CO₂(CH₂)_{d}(CH₂CH₂O)ₑR²⁶, or —(CH₂)ₐ(CH₂CH₂O)_{b}(CH₂)_{c}OC(O)NR¹⁹CO₂(CH₂)_{d}(CH₂CH₂O)ₑR²⁷; —(CH₂)_{c}OR⁶⁸, —CH₂(CHOH)_{c}R⁶⁹, —CH₂(CHOH)_{c}CO₂H, —(CHCO₂H)_{c}CO₂H, —(CH₂)_{c}NR⁷⁰R⁷¹, —CH[(CH₂)_{f}NH₂]_{c}CO₂H, —CH[(CH₂)_{f}NH₂]_{c}CH₂OH, —CH₂(CHNH₂)_{c}CH₂NR⁷²R⁷³, —(CH₂CH₂O)ₑR⁷⁴, —(CH₂)ₜCO(CH₂CH₂O)ₑR⁷⁵, —(CH₂)ᵤ(CH₂CH₂O)ⱼ(CH₂)ₖNR⁵⁸C(O)NR⁵⁹(CH₂)ₗ(CH₂CH₂O)ₒR⁷⁶, —(CH₂)ᵤ(CH₂CH₂O)ⱼ(CH₂)ₖNR⁶⁰C(S)NR⁶¹(CH₂)ₗ(CH₂CH₂O)ₒR⁷⁷, —(CH₂)ᵤ(CH₂CH₂O)ⱼ(CH₂)ₖC(O)NR⁶²(CH₂)ₗ(CH₂CH₂O)ₒR⁷⁸, —(CH₂)ᵤ(CH₂CH₂O)ⱼ(CH₂)ₖS(O)₂NR⁶³(CH₂)ₗ(CH₂CH₂O)ₒR⁷⁹, —(CH₂)ᵤ(CH₂CH₂O)ⱼ(CH₂)ₖNR⁶⁴S(O)₂(CH₂)ₗ(CH₂CH₂O)ₒR⁸⁰, —(CH₂)ᵤ(CH₂CH₂O)ⱼ(CH₂)ₖNR⁶⁵C(O)(CH₂)ₗ(CH₂CH₂O)ₒR⁸¹, —(CH₂)ᵤ(CH₂CH₂O)ⱼ(CH₂)ₖNR⁶⁶C(O)O(CH₂)ₗ(CH₂CH₂O)ₒR⁸², or —(CH₂)ᵤ(CH₂CH₂O)ⱼ(CH₂)ₖOC(O)NR⁶⁷(CH₂)ₗ(CH₂CH₂O)ₒR⁸³, —(CH₂)ₐSO₃H, —(CH₂)ₐSO₃⁻, —(CH₂)ₐOSO₃H, —(CH₂)ₐOSO₃⁻, —(CH₂)ₐNHSO₃H, —(CH₂)ₐNHSO₃⁻, —(CH₂)ₐPO₃H₂, —CH₂)ₐ PO₃H⁻, —(CH₂)ₐPO₃²⁻, —(CH₂)ₐOPO₃H₂, —(CH₂)ₐOPO₃H⁻, or —(CH₂)ₐOPO³.

Each of R², R¹⁰, R¹¹, R¹², R¹³, R¹⁴, R¹⁵, R¹⁶, R¹⁷, R¹⁸, R¹⁹, R⁵⁸, R⁵⁹, R⁶⁰, R⁶¹, R⁶², R⁶³, R⁶⁴, R⁶⁵, R⁶⁶ and R⁶⁷ is independently —H or —CH₃.

Each of R²⁰, R²¹, R²², R²³, R²⁴, R²⁵, R²⁶, and R²⁷ is independently —H, —CH₃, —(CH₂)_{f}NR²⁸C(O)NR²⁹(CH₂)_{g}(CH₂CH₂O)ₕR³⁸, —(CH₂)_{f}NR³⁰CSNR³¹(CH₂)_{g}(CH₂CH₂O)ₕR³⁹, —(CH₂)_{f}(C(O)NR³²(CH₂)_{g}(CH₂CH₂O)ₕR⁴⁰, —(CH₂)_{f}S(O)₂NR³³(CH₂)_{g}(CH₂CH₂O)ₕR⁴¹, —(CH₂)_{f}NR³⁴S(O)₂(CH₂)_{g}(CH₂CH₂O)ₕR⁴², —(CH₂)_{f}NR³⁵C(O)(CH₂)_{g}(CH₂CH₂O)ₕR⁴³, —(CH₂)_{f}NR³⁶C(O)O(CH₂)_{g}(CH₂CH₂O)ₕR⁴⁴, —(CH₂)_{f}OC(O)NR³⁷(CH₂)_{g}(CH₂CH₂O)ₕR⁴⁵, —CO(AA), or —CONH(PS).

each of R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷ and R⁴⁸ is independently —H or —CH₃;

Each of R⁶⁸, R⁶⁹, R⁷⁰, R⁷¹, R⁷², R⁷³, R⁷⁴, R⁷⁵, R⁷⁶, R⁷⁷, R⁷⁸, R⁷⁹, and R⁸⁰ is independently —H, —CH₃, —(CH₂)ₚS(O)₂NR⁸⁴(CH₂)_{q}(CH₂CH₂O)ₛR⁸¹, —(CH₂)ₚNR⁸⁵S(O)₂(CH₂)_{q}(CH₂CH₂O)ₛR⁸³, —(CH₂)ₚNR⁸⁶C(O)(CH₂)_{q}(CH₂CH₂O)ₛR⁸⁵, —(CH₂)ₚNR⁸⁶C(O)O(CH₂)_{q}(CH₂CH₂O)ₛR⁸⁷, or —(CH₂)ₚOC(O)NR⁸⁸(CH₂)_{q}(CH₂CH₂O)ₛR⁸⁹.

Each of R⁸¹, R⁸², R⁸³, R⁸⁴, R⁸⁵, R⁸⁶, R⁸⁷, R⁸⁸, and R⁸⁹ is independently —H or —CH₃.

AA is as defined above.

Each (PS) is independently a sulfated or non-sulfated polysaccharide chain comprising one or more monosaccharide units connected by glycosidic linkages. The polysaccharide chain (PS) may be any appropriate length. For instance, in some embodiments, the polysaccharide chain may include 1 to 100 monosaccharide unit(s), 1 to 90 monosaccharide unit(s), 1 to 80 monosaccharide unit(s), 1 to 70 monosaccharide unit(s), 1 to 60 monosaccharide unit(s), 1 to 50 monosaccharide unit(s), 1 to 40 monosaccharide unit(s), 1 to 30 monosaccharide unit(s), 1 to 20 monosaccharide unit(s), or even 1 to 10 monosaccharide unit(s). In some embodiments, the polysaccharide chain (PS) is a homopolysaccharide chain consisting of either pentose or hexose monosaccharide units. In other embodiments, the polysaccharide chain (PS) is a heteropolysaccharide chain consisting of one or both pentose and hexose monosaccharide units. In some embodiments, the monosaccharide units of the polysaccharide chain (PS) are selected from glucose, fructose, mannose, xylose and ribose. In some embodiments, the polysaccharide chain (PS) refers to a single monosaccharide unit (e.g., either glucose or fructose).

Each of t and u is independently 1, 2, 3, 4, or 5.

Each of a, d, g, l, and q is independently 0, 1, 2, 3, 4, 5, 6, 7, 8, 9 or 10.

Each of c, f, k, and p is independently 0, 1, 2, 3, 4, 5, 6, 7, 8, 9 or 10.

Each of b, j, e, h, o, and s is independently 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100.

PG is a protecting group. Generally, the protecting group may be any suitable protecting group known in the art. The protecting group may suitably be, without limitation, benzyl (“Bn”), t-butyl, p-t-butylbenzyl, methyl, p-methoxybenzyl, p-sec-butyl, p-i-propylbenzyl, p-n-propylbenzyl, p-ethylbenzyl, or p-methylbenzyl.

In some embodiments, the protecting group protects a single amino acid. In some embodiments, the amino acid is aspartic acid, asparagine, arginine, histidine, lysine, glutamic acid, glutamine, serine, or homoserine. In some embodiments, the amino acid is aspartic acid or serine. In some embodiments, the amino acid is D-serine having a Bn protecting group.

Generally, the reactant may be present in any suitable amount known in the art. In some embodiments, the reactant is present in an amount of from about 0.001 g/mL to about 1 g/mL. In some embodiments, the reactant is present in an amount of from about 0.0067 g/mL to about 0.04 g/mL.

Generally, the organic solvent may be any suitable organic solvent known in the art. In some embodiments, the organic solvent comprises a solvent selected from alcohols, methanol, ethanol, propanol, isopropanol, butanol, sec-butanol, t-butanol, acetates, methyl acetate, ethyl acetate, acetic acid, n-propionic acid, n-butanoic acid, iso-butyric acid, and combinations thereof.

In some embodiments, the mixture is substantially free of aqueous solvents. In some embodiments, the mixture is substantially free of water. In some embodiments, the mixture does not comprise water.

In some embodiments, the mixture further comprises an aqueous solvent. In some embodiments, the mixture further comprises water.

In some embodiments, the solvent comprises organic solvent and aqueous solvent in a ratio of from about 100:0 to about 50:50. In some embodiments, the solvent is 100% organic solvent.

Generally, the hydrogenation agent may be any suitable hydrogenation agent known in the art. In some embodiments, the hydrogenation agent comprises a hydrogenation agent selected from hydrogenation hydrogen sources, hydrogen (H₂), formic acid, formate, isopropanol, dihydroanthracene, 1,4-cyclohexadiene, 1,3-cyclohexadiene, 1-methyl-1,4-cyclohexadiene, and combinations thereof.

Generally, the hydrogenation agent may be present in any suitable amount known in the art. In some embodiments, the hydrogenation agent is present in an amount of from about 1 molar equivalent to hydrogen to an excess amount relevant to hydrogen. In some embodiments, the hydrogenation agent is present in an amount of from about 20 molar equivalents to hydrogen to about 30 molar equivalents to hydrogen.

Generally, the catalyst may be any suitable catalyst known in the art. In some embodiments, the catalyst comprises a catalyst selected from hydrogenation catalysts, noble metal catalysts, palladium (Pd), platinum (Pt), gold (Au), rhodium (Rh), ruthenium (Ru), silver (Ag), osmium (Os), iridium (Ir), transition metal catalysts, nickel (Ni), Raney nickel, Urushibara nickel, iron (Fe), molybdenum (Mo), cobalt (Co), copper (Cu), chromium (Cr), catalysts supported on a catalyst support, catalysts supported on carbon, catalysts supported on alumina, catalysts supported on silica, Pd/C, Pt/C, Pd/C containing water in an amount in a range of from about 1% to about 50%, Pd/C containing about 50% water, and combinations thereof.

Generally, the catalyst may be present in any suitable amount known in the art. In some embodiments, the catalyst is present in an amount of from about 1 wt % to about 30 wt %. In some embodiments, the catalyst is present in an amount of from about 5 wt % to about 20 wt %.

Generally, forming the mixture may occur according to any suitable formation process known in the art. In some embodiments, the mixture components are concurrently added to form the mixture. In some embodiments, the mixture components are individually added to form the mixture. In some embodiments, the mixture components are individually added in any suitable combination to form the mixture.

Generally, reacting the mixture may occur at any suitable reaction temperature known in the art. In some embodiments, the reacting the mixture occurs at a reaction temperature in the range of about −20° C. to about 100° C.

Generally, reacting the mixture may occur at any suitable reaction pressure known in the art. In some embodiments, reacting the mixture occurs at atmospheric pressure. In some embodiments, reacting the mixture occurs at an elevated reaction pressure. In some embodiments, reacting the mixture occurs at a reaction pressure in the range of about 1 PSIG to about 25 PSIG. In some embodiments, reacting the mixture occurs at a reaction pressure greater than about 25 PSIG. In some embodiments, reacting the mixture occurs at a reaction pressure in the range of about 1 PSIG to about 50 PSIG.

Generally, reacting the mixture may occur for any suitable reaction time known in the art. In some embodiments, reacting the mixture occurs during a reaction time in the range of about 1 hour to about 48 hours.

Generally, reacting the mixture may occur in any suitable reactor known in the art. In some embodiments, reacting the mixture occurs in a reactor selected from glass reactors, glass-lined steel reactors, stainless steel reactors, glass-lined stainless-steel reactors, steel reactors having disposable liners, stainless steel reactors having disposable liners, steel reactors having polymeric liners, stainless steel reactors having polymeric liners, Hastelloy reactors, atmospheric pressure reactors, pressure reactors, and combinations thereof.

Generally, the method may also include any further suitable processing steps known in the art. In some embodiments, the method further includes a method step selected from filtering, centrifuging, centrifugal filtration, isolating, washing, drying, concentrating, catalyst scavenging, purifying, and combinations thereof.

In some embodiments, the method further includes purifying the compound of Formula II or a salt thereof with a first solvent and an anti-solvent, and optionally removing the first solvent from the compound of Formula II or a salt thereof.

In some embodiments, the first solvent is selected from organic solvents, dimethyl sulfoxide (DMSO), and combinations thereof. In some embodiments, the anti-solvent is selected from acetates, ethyl acetate, isopropyl acetate, and combinations thereof.

The present disclosure is also generally directed to a method of purifying a compound of Formula II or a salt thereof. In some embodiments, the method includes precipitating a compound of Formula II or a salt thereof with a first solvent and an anti-solvent, and optionally removing the first solvent from the compound of Formula II or a salt thereof.

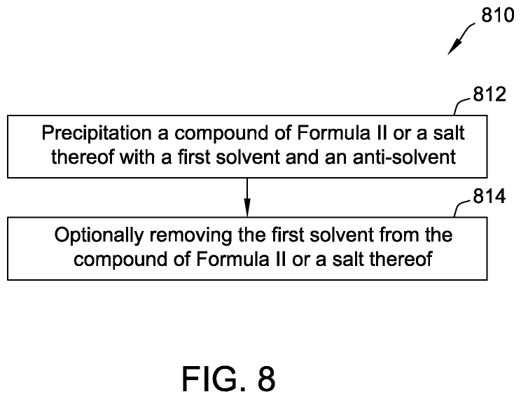

FIG. 8 is an exemplary method flow chart 810. In this exemplary embodiment, method flow chart 810 depicts the essential method steps of the method embodiments described herein and is not intended to limit the method embodiments. Method step 812 includes precipitating a compound of Formula II or a salt thereof with a first solvent and an anti-solvent. Method step 814 includes optionally removing the first solvent from the compound of Formula II or a salt thereof.

In some embodiments, removing the first solvent from the compound of Formula II or a salt thereof includes removing the first solvent from the compound of Formula II or a salt thereof with a solvent precipitation process including washing the compound of Formula II or a salt thereof with the first solvent, and precipitating the compound of Formula II or a salt thereof by mixing the washed compound of Formula II or a salt thereof with a second solvent.

In some embodiments, the first solvent is selected from organic solvents, dimethyl sulfoxide (DMSO), and combinations thereof. In some embodiments, the second solvent is selected from aqueous solvents, water, and combinations thereof.

In some embodiments, a compound of Formula I or a salt thereof and a compound of Formula II or a salt thereof may each independently have a molecular weight of no more than 20000. In some such aspects, the molecular weight is no more than 15000, 14000, 13000, 12000, 11000, 10000, 9000, 8000, 7000, 6000, 5000, 4500, 4000, 3500, 3000, 2500, 2000, 1500, 1000, 900, 800, 700, 600, 500, 400 or 300. In other aspects, a compound of Formula I or a salt thereof and a compound of Formula II or a salt thereof may each independently have a molecular weight that is greater than 20000. In some embodiments, the molecular weight is suitably from about 300 to about 1000, or from about 300 to about 750. In some embodiments, a compound of Formula II or a salt thereof has a molecular weight of from about 300 to about 600, or from about 300 to about 500.

In some embodiments, the reaction yield is at least 70%. In some embodiments, the reaction yield is at least 75%. In some embodiments, the reaction yield is at least 80%. In some embodiments, the reaction yield is at least 85%. In some embodiments, the reaction yield is at least 90%. In some embodiments, the reaction yield is at least 95%. In some embodiments, the reaction yield is at least 97%. In some embodiments, the reaction yield is at least 99%.

In some embodiments, the purity of the reaction product is at least 70%. In some embodiments, the purity of the reaction product is at least 75%. In some embodiments, the purity of the reaction product is at least 80%. In some embodiments, the purity of the reaction product is at least 85%. In some embodiments, the purity of the reaction product is at least 90%. In some embodiments, the purity of the reaction product is at least 95%. In some embodiments, the purity of the reaction product is at least 97%. In some embodiments, the purity of the reaction product is at least 99%.

In one aspect, (2R,2′R)-2,2′-((3,6-diaminopyrazine-2,5-dicarbonyl)bis(azanediyl))bis(3-hydroxypropanoic acid) is prepared according to a method represented by FIG. 3.

The embodiments of this disclosure include:

Embodiment 1. A method of preparing a compound of Formula II or a salt thereof,

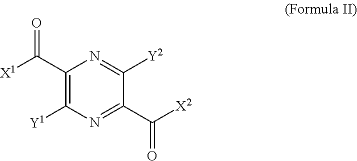

the method comprising:
forming a mixture comprising
   a compound of Formula I or a salt thereof,

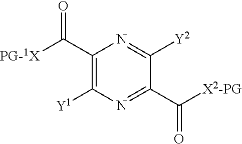

wherein:
PG is a protecting group;
each of Y¹ and Y² is independently selected from hydrogen, halogen, amine, nitroso, nitro, amide, ester, and carboxyl; and
each of X¹ and X² is independently selected from
   (i) one or more natural or unnatural α-amino acids or a polypeptide chain that includes one or more natural or unnatural α-amino acids linked together by peptide bonds, or
   (ii) N(R¹)(R²) wherein:
   each R¹ is independently —(CH₂)ₐ(CH₂CH₂O)_{b}(CH₂)_{c}NR¹⁰CONR¹¹(CH₂)_{d}(CH₂CH₂O)ₑR²⁰, —(CH₂)ₐ(CH₂CH₂O)_{b}(CH2)_{c}NR¹²CSNR¹³(CH₂)_{d}(CH₂CH₂O)ₑR²¹, —(CH₂)ₐ(CH₂CH₂O)_{b}(CH₂)_{c}CONR¹⁴(CH₂)_{d}(CH₂CH₂O)ₑR²², —(CH₂)ₐ(CH₂CH₂O)_{b}(CH₂)_{c}NR¹⁵SO₂(CH₂)_{d}(CH₂CH₂O)ₑR²³, —(CH₂)ₐ(CH₂CH₂O)_{b}(CH₂)_{c}SO₂NR¹⁶(CH₂)_{d}(CH₂CH₂O)ₑR²⁴, —(CH2)ₐ(CH₂CH₂O)_{b}(CH₂)_{c}NR¹⁷CO(CH₂)_{d}(CH₂CH₂O)ₑR²⁵, —(CH2)ₐ(CH₂CH₂O)_{b}(CH₂)_{c}NR¹⁸CO₂(CH₂)_{d}(CH₂CH₂O)ₑR²⁶, or —(CH₂)ₐ(CH₂CH₂O)_{b}(CH₂)_{c}OC(O)NR¹⁹CO₂(CH₂)_{d}(CH₂CH₂O)ₑR²⁷; —(CH₂)₂OR⁶⁸, —CH₂(CHOH)_{c}R⁶⁹, —CH₂(CHOH)_{c}CO₂H, —(CHCO₂H)_{c}CO₂H, —(CH₂)_{c}NR⁷⁰R⁷¹, —CH[(CH₂)_{f}NH₂]_{c}CO₂H, —CH[(CH₂)_{f}NH₂]_{c}CH₂OH, —CH₂(CHNH₂)_{c}CH2NR⁷²R⁷³, —(CH₂CH₂O)ₑR⁷⁴, —(CH₂)ₜCO(CH₂CH₂O)ₑR⁷⁵, —(CH₂)ᵤ(CH₂CH₂O)ⱼ(CH₂)ₖNR⁵⁸C(O)NR⁵⁹(CH₂)ₗ(CH₂CH₂O)ₒR⁷⁶, —(CH₂)ᵤ(CH₂CH₂O)ⱼ(CH₂)ₖNR⁶⁰C(S)NR⁶¹(CH₂)ₗ(CH₂CH₂O)ₒR⁷⁷, —(CH₂)ᵤ(CH₂CH₂O)ⱼ(CH₂)ₖC(O)NR⁶²(CH₂)ₗ(CH₂CH₂O)ₒR⁷⁸, —(CH₂)ᵤ(CH₂CH₂O)ⱼ(CH₂)ₖS(O)₂NR⁶³(CH₂)ₗ(CH₂CH₂O)ₒR⁷⁹, —(CH₂)ᵤ(CH₂CH₂O)ⱼ(CH₂)ₖNR⁶⁴S(O)₂(CH₂)ₗ(CH₂CH₂O)ₒR⁸⁰, —(CH₂)ᵤ(CH₂CH₂O)ⱼ(CH₂)ₖNR⁶⁵C(O)(CH₂)ₗ(CH₂CH₂O)ₒR⁸¹, —(CH₂)ᵤ(CH₂CH₂O)ⱼ(CH₂)ₖNR⁶⁶C(O)O(CH₂)ₗ(CH₂CH₂O)ₒR⁸², or —(CH₂)ᵤ(CH₂CH₂O)ⱼ(CH₂)ₖOC(O)NR⁶⁷(CH₂)ₗ(CH₂CH₂O)ₒR⁸³, —(CH₂)ₐSO₃H, —(CH₂)ₐSO₃⁻, —(CH₂)ₐOSO₃H, —(CH₂)ₐOSO₃⁻, —(CH₂)ₐNHSO₃H, —(CH₂)ₐNHSO₃⁻, —(CH₂)ₐPO₃H₂, —(CH₂)ₐPO₃H⁻, —(CH₂)ₐPO₃²⁻, —(CH₂)ₐOPO₃H₂, —(CH₂)ₐOPO₃H⁻, or —(CH₂)ₐOPO³;
   each of R², R¹⁰, R¹¹, R¹², R¹³, R¹⁴, R¹⁵, R¹⁶, R¹⁷, R¹⁸, R¹⁹, R⁵⁸, R⁵⁹, R⁶⁰, R⁶¹, R⁶², R⁶³, R⁶⁴, R⁶⁵, R⁶⁶ and R⁶⁷ is independently —H or —CH₃;
   each of R²⁰, R²¹, R²², R²³, R²⁴, R²⁵, R²⁶, and R²⁷ is independently —H, —CH₃, —(CH₂)_{f}NR²⁸C(O)NR²⁹(CH₂)_{g}(CH₂CH₂O)NR³⁸, —(CH₂)_{f}NR³⁰CSNR³¹(CH₂)_{g}(CH₂CH₂O)ₕR³⁹, —(CH₂)_{f}C(O)NR³²(CH₂)_{g}(CH₂CH₂O)ₕR⁴⁰, —(CH₂)_{f}S(O)₂NR³³(CH₂)_{g}(CH₂CH₂O)ₕR⁴¹, —(CH₂)_{f}NR³⁴S(O)₂(CH₂)_{g}(CH₂CH₂O)ₕR⁴², —(CH₂)_{f}NR³⁵C(O)(CH₂)_{g}(CH₂CH₂O)ₕR⁴³, —(CH₂)_{f}NR³⁶C(O)O(CH₂)_{g}(CH₂CH₂O)ₕR⁴⁴, —(CH₂)_{f}OC(O)NR³⁷(CH₂)_{g}(CH₂CH₂O)ₕR⁴⁵, —CO(AA), or —CONH(PS);
   each of R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷ and R⁴⁸ is independently —H or —CH₃;
   each of R⁶⁸, R⁶⁹, R⁷⁰, R⁷¹, R⁷², R⁷³, R⁷⁴, R⁷⁵, R⁷⁶, R⁷⁷, R⁷⁸, R⁷⁹, and R⁸⁰ is independently —H, —CH₃, —(CH₂)ₚS(O)₂NR⁸⁴(CH₂)_{q}(CH₂CH₂O)ₛR⁸¹, —(CH₂)ₚNR⁸⁵S(O)₂(CH₂)_{q}(CH₂CH₂O)ₛR⁸³, —(CH₂)ₚNR⁸⁶C(O)(CH₂)_{q}(CH₂CH₂O)ₛR⁸⁵, —(CH₂)ₚNR⁸⁶C(O)O(CH₂)_{q}(CH₂CH₂O)ₛR⁸⁷, or —(CH₂)ₚOC(O)NR⁸⁸(CH₂)_{q}(CH₂CH₂OₛR⁸⁹;
   each of R⁸¹, R⁸², R⁸³, R⁸⁴, R⁸⁵, R⁸⁶, R⁸⁷, R⁸⁸, and R⁸⁹ is independently —H or —CH₃;
   each (AA) is independently one or more natural or unnatural α-amino acids or a polypeptide chain that includes one or more natural or unnatural α-amino acids linked together by peptide bonds;
   each (PS) is independently a sulfated or non-sulfated polysaccharide chain comprising one or more monosaccharide units connected by glycosidic linkages;
   each of t and u is independently 1, 2, 3, 4, or 5;
   each of a, d, g, l, and q is independently 0, 1, 2, 3, 4, 5, 6, 7, 8, 9 or 10;
   each of c, f, k, and p is independently 0, 1, 2, 3, 4, 5, 6, 7, 8, 9 or 10; and
   each of b, j, e, h, o, and s is independently 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100;
      an organic solvent;
      a catalyst; and
      a hydrogenation agent; and
reacting the mixture.

Embodiment 2. The method of embodiment 1, wherein each of X¹ and X² is D-serine, each of Y¹ and Y² is amine, PG is a benzyl protecting group, and the compound of Formula I is dibenzyl 2,2′-((3,6-diaminopyrazine-2,5-dicarbonyl)bis(azanediyl))(2R,2′R)-bis(3-hydroxypropanoate) of the following structure:

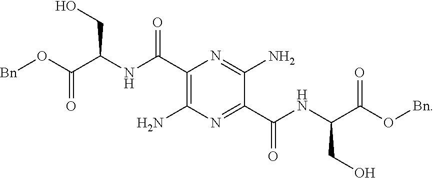 [SMILES: Nc1nc(C(=O)N[C@H](CO)C(=O)OCc2ccccc2)c(N)nc1C(=O)N[C@H](CO)C(=O)OCc1ccccc1]

Embodiment 3. The method of embodiment 1, wherein each of X¹ and X² is D-serine, each of Y¹ and Y² is amine, and the compound of Formula II is (2R,2′R)-2,2′-((3,6-diaminopyrazine-2,5-dicarbonyl)bis(azanediyl))bis(3-hydroxypropanoic acid) of the following structure:

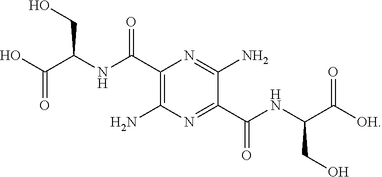 [SMILES: Nc1nc(C(=O)N[C@H](CO)C(=O)O)c(N)nc1C(=O)N[C@H](CO)C(=O)O]

Embodiment 4. The method of embodiment 1, wherein PG is a protecting group selected from benzyl (“Bn”), t-butyl, p-t-butylbenzyl, methyl, p-methoxybenzyl, p-sec-butyl, p-i-propylbenzyl, p-n-propylbenzyl, p-ethylbenzyl, and p-methylbenzyl.

Embodiment 5. The method of embodiment 1, wherein the organic solvent comprises a solvent selected from alcohols, methanol, ethanol, propanol, isopropanol, butanol, sec-butanol, t-butanol, acetates, methyl acetate, ethyl acetate, acetic acid, n-propionic acid, n-butanoic acid, iso-butyric acid, and combinations thereof.

Embodiment 6. The method of embodiment 1, wherein the hydrogenation agent comprises a hydrogenation agent selected from hydrogenation hydrogen sources, hydrogen (H₂), formic acid, formate, isopropanol, dihydroanthracene, 1,4-cyclohexadiene, 1,3-cyclohexadiene, 1-methyl-1,4-cyclohexadiene, and combinations thereof.

Embodiment 7. The method of embodiment 1, wherein the catalyst comprises a catalyst selected from hydrogenation catalysts, noble metal catalysts, palladium (Pd), platinum (Pt), gold (Au), rhodium (Rh), ruthenium (Ru), silver (Ag), osmium (Os), iridium (Ir), transition metal catalysts, nickel (Ni), Raney nickel, Urushibara nickel, iron (Fe), molybdenum (Mo), cobalt (Co), copper (Cu), chromium (Cr), catalysts supported on a catalyst support, catalysts supported on carbon, catalysts supported on alumina, catalysts supported on silica, Pd/C, Pt/C, Pd/C containing water in an amount in a range of from about 1% to about 50%, Pd/C containing about 50% water, and combinations thereof.

Embodiment 8. The method of embodiment 1, wherein the method step of reacting the mixture occurs at a reaction temperature in the range of about −20° C. to about 100° C.

Embodiment 9. The method of embodiment 1, wherein the method step of reacting the mixture occurs at atmospheric pressure.

Embodiment 10. The method of embodiment 1, wherein the method step of reacting the mixture occurs at an elevated reaction pressure.

Embodiment 11. The method of embodiment 1, wherein the method step of reacting the mixture occurs at a reaction pressure in the range of about 1 PSIG to about 50 PSIG.

Embodiment 12. The method of embodiment 1, wherein the method step of reacting the mixture occurs during a reaction time in the range of about 1 hour to about 48 hours.

Embodiment 13. The method of embodiment 1, wherein the mixture is substantially free of aqueous solvents.

Embodiment 14. The method of embodiment 1, wherein the mixture further comprises an aqueous solvent.

Embodiment 15. The method of embodiment 1, further comprising a method step selected from filtering, centrifuging, centrifugal filtration, isolating, washing, drying, concentrating, catalyst scavenging, purifying, and combinations thereof.

Embodiment 16. The method of embodiment 1, further comprising:
purifying the compound of Formula II or a salt thereof with a first solvent and an anti-solvent; and
optionally removing the first solvent from the compound of Formula II or a salt thereof.

Embodiment 17. The method of embodiment 16, wherein:
the first solvent is selected from organic solvents, dimethyl sulfoxide (DMSO), and combinations thereof; and
the anti-solvent is selected from acetates, ethyl acetate, isopropyl acetate, and combinations thereof.

Embodiment 18. A method of purifying a compound of Formula II or a salt thereof,

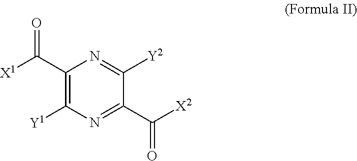

the method comprising:
precipitating the compound of Formula II or a salt thereof with a first solvent and an anti-solvent; and
optionally removing the first solvent from the compound of Formula II or a salt thereof;
wherein:
each of Y¹ and Y² is independently selected from hydrogen, halogen, amine, nitroso, nitro, amide, ester, and carboxyl; and
each of X¹ and X² is independently selected from
   (i) one or more natural or unnatural α-amino acids or a polypeptide chain that includes one or more natural or unnatural α-amino acids linked together by peptide bonds, or
   (ii) N(R¹)(R²) wherein:
   each R¹ is independently —(CH₂)ₐ(CH₂CH₂O)_{b}(CH₂)_{c}NR¹⁰CONR¹¹(CH₂)_{d}(CH₂CH₂O)ₑR²⁰, —(CH₂)ₐ(CH₂CH₂O)_{b}(CH₂)_{c}NR¹²CSNR¹³(CH₂)_{d}(CH₂CH₂O)ₑR²¹, —(CH₂)ₐ(CH₂CH₂O)_{b}(CH₂)_{c}CONR¹⁴(CH₂)_{d}(CH₂CH₂O)ₑR²², —(CH₂)ₐ(CH₂CH₂O)_{b}(CH₂)_{c}NR¹⁵SO₂(CH₂)_{d}(CH₂CH₂O)ₑR²³, —(CH₂)ₐ(CH₂CH₂O)_{b}(CH₂)_{c}SO₂NR¹⁶(CH₂)_{d}(CH₂CH₂O)ₑR²⁴, —(CH2)ₐ(CH₂CH₂O)_{b}(CH₂)_{c}NR¹⁷CO(CH₂)_{d}(CH₂CH₂O)ₑR²⁵, —(CH2)ₐ(CH₂CH₂O)_{b}(CH₂)_{c}NR¹⁸CO₂(CH₂)_{d}(CH₂CH₂O)ₑR^{26,} or —(CH₂)ₐ(CH₂CH₂O)_{b}(CH₂)_{c}OC(O)NR¹⁹CO₂(CH₂)_{d}(CH₂CH₂O)ₑR²⁷; —(CH₂)_{c}OR⁶⁸, —CH₂(CHOH)_{c}R⁶⁹, —CH₂(CHOH)_{c}CO₂H, —(CHCO₂H)_{c}CO₂H, —(CH₂)_{c}NR⁷⁰R⁷¹, —CH[(CH₂)_{f}NH₂]_{c}CO₂H, —CH[(CH₂)_{f}NH₂]_{c}CH₂OH, —CH₂(CHNH₂)_{c}CH2NR⁷²R⁷³, —(CH₂CH₂O)ₑR⁷⁴, —(CH₂)ₜCO(CH₂CH₂O)ₑR⁷⁵, —(CH₂)ᵤ(CH₂CH₂O)ⱼ(CH₂)ₖNR⁵⁸C(O)NR⁵⁹(CH₂)ₗ(CH₂CH₂O)ₒR⁷⁶, —(CH₂)ᵤ(CH₂CH₂O)ⱼ(CH₂)ₖNR⁶⁰C(S)NR⁶¹(CH₂)ₗ(CH₂CH₂O)ₒR⁷⁷, —(CH₂)ᵤ(CH₂CH₂O)ⱼ(CH₂)ₖC(O)NR⁶²(CH₂)ₗ(CH₂CH₂O)ₒR⁷⁸, —(CH₂)ᵤ(CH₂CH₂O)ⱼ(CH₂)ₖS(O)₂NR⁶³(CH₂)ₗ(CH₂CH₂O)ₒR⁷⁹, —(CH₂)ᵤ(CH₂CH₂O)ⱼ(CH₂)ₖNR⁶⁴S(O)₂(CH₂)ₗ(CH₂CH₂O)ₒR⁸⁰, —(CH₂)ᵤ(CH₂CH₂O)ⱼ(CH₂)ₖNR⁶⁵C(O)(CH₂)ₗ(CH₂CH₂O)ₒR⁸¹, —(CH₂)ᵤ(CH₂CH₂O)ⱼ(CH₂)ₖNR⁶⁶C(O)O(CH₂)ₗ(CH₂CH₂O)ₒR⁸², or —(CH₂)ᵤ(CH₂CH₂O)ⱼ(CH₂)ₖOC(O)NR⁶⁷(CH₂)ₗ(CH₂CH₂O)ₒR⁸³, —(CH₂)ₐSO₃H, —(CH₂)ₐSO₃⁻, —(CH₂)ₐOSO₃H, —(CH₂)ₐOSO₃⁻, —(CH₂)ₐNHSO₃H, —(CH₂)ₐNHSO₃⁻, —(CH₂)ₐPO₃H₂, —(CH₂)ₐPO₃H⁻, —(CH₂)ₐPO₃²⁻, —(CH₂)ₐOPO₃H₂, —(CH₂)ₐOPO₃H⁻, or —(CH₂)ₐOPO³;
   each of R², R¹⁰, R¹¹, R¹², R¹³, R¹⁴, R¹⁵, R¹⁶, R¹⁷, R¹⁸, R¹⁹, R⁵⁸, R⁵⁹, R⁶⁰, R⁶¹, R⁶², R⁶³, R⁶⁴, R⁶⁵, R⁶⁶ and R⁶⁷ is independently —H or —CH₃;
   each of R²⁰, R²¹, R²², R²³, R²⁴, R²⁵, R²⁶, and R²⁷ is independently —H, —CH₃, —(CH₂)_{f}NR²⁸C(O)NR²⁹(CH₂)_{g}(CH₂CH₂O)ₕR³⁸, —(CH₂)_{f}NR³⁰CSNR³¹(CH₂)_{g}(CH₂CH₂O)ₕR³⁹, —(CH₂)_{f}C(O)NR³²(CH₂)_{g}(CH₂CH₂O)ₕR⁴⁰, —(CH₂)_{f}S(O)₂NR³³(CH₂)_{g}(CH₂CH₂O)ₕR⁴¹, —(CH₂)_{f}NR³⁴S(O)₂(CH₂)_{g}(CH₂CH₂O)ₕR⁴², —(CH₂)_{f}NR³⁵C(O)(CH₂)_{g}(CH₂CH₂O)ₕR⁴³, —(CH₂)_{f}NR³⁶C(O)O(CH₂)_{g}(CH₂CH₂O)ₕR⁴⁴, —(CH₂)_{f}OC(O)NR³⁷(CH₂)_{g}(CH₂CH₂O)ₕR⁴⁵, —CO(AA), or —CONH(PS);
   each of R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷ and R⁴⁸ is independently —H or —CH₃;
   each of R⁶⁸, R⁶⁹, R⁷⁰, R⁷¹, R⁷², R⁷³, R⁷⁴, R⁷⁵, R⁷⁶, R⁷⁷, R⁷⁸, R⁷⁹, and R⁸⁰ is independently —H, —CH₃, —(CH₂)ₚS(O)₂NR⁸⁴(CH₂)_{q}(CH₂CH₂O)ₛR⁸¹, —(CH₂)ₚNR⁸⁵S(O)₂(CH₂)_{q}(CH₂CH₂O)ₛR⁸³, —(CH₂)ₚNR⁸⁶C(O)(CH₂)_{q}(CH₂CH₂O)ₛR⁸⁵, —(CH₂)ₚNR⁸⁶C(O)O(CH₂)_{q}(CH₂CH₂O)ₛR⁸⁷, or —(CH₂)ₚOC(O)NR⁸⁸(CH₂)_{q}(CH₂CH₂O)ₛR⁸⁹;
   each of R⁸¹, R⁸², R⁸³, R⁸⁴, R⁸⁵, R⁸⁶, R⁸⁷, R⁸⁸, and R⁸⁹ is independently —H or —CH₃;
   each (AA) is independently one or more natural or unnatural α-amino acids or a polypeptide chain that includes one or more natural or unnatural α-amino acids linked together by peptide bonds;
   each (PS) is independently a sulfated or non-sulfated polysaccharide chain comprising one or more monosaccharide units connected by glycosidic linkages;
   each of t and u is independently 1, 2, 3, 4, or 5;
   each of a, d, g, l, and q is independently 0, 1, 2, 3, 4, 5, 6, 7, 8, 9 or 10;
   each of c, f, k, and p is independently 0, 1, 2, 3, 4, 5, 6, 7, 8, 9 or 10; and
   each of b, j, e, h, o, and s is independently 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100;

Embodiment 19. The method of embodiment 18, wherein removing the first solvent from the compound of Formula II or a salt thereof comprises removing the first solvent from the compound of Formula II or a salt thereof with a solvent precipitation process comprising:
washing the compound of Formula II or a salt thereof with the first solvent; and
precipitating the compound of Formula II or a salt thereof by mixing the washed compound of Formula II or a salt thereof with a second solvent.

Embodiment 20. The method of embodiment 19, wherein:
the first solvent is selected from organic solvents, dimethyl sulfoxide (DMSO), and combinations thereof; and
the second solvent is selected from aqueous solvents, water, and combinations thereof.

### EXAMPLES

Without further elaboration, it is believed that one skilled in the art using the preceding description can utilize the present invention to its fullest extent. The following Examples are, therefore, to be construed as merely illustrative, and not limiting of the disclosure in any way whatsoever. The starting material for the following Examples may not have necessarily been prepared by a particular preparative run whose procedure is described in other Examples. It also is understood that any numerical range recited herein includes all values from the lower value to the upper value. For example, if a range is stated as 10-50, it is intended that values such as 12-30, 20-40, or 30-50, etc., are expressly enumerated in this specification. These are only examples of what is specifically intended, and all possible combinations of numerical values between and including the lowest value and the highest value enumerated are to be considered to be expressly stated in this application.

The following definitions are used in these examples:

MB-102: (2R,2′R)-2,2′-((3,6-diaminopyrazine-2,5-dicarbonyl)bis(azanediyl))bis(3-hydroxypropanoic acid).

MB-102 int: an MB-102 impurity; partially de-protected benzyl 2,2′-((3,6-diaminopyrazine-2,5-dicarbonyl)bis(azanediyl))(2R,2′R)-bis(3-hydroxypropanoate)

MP-3269: dibenzyl 2,2′-((3,6-diaminopyrazine-2,5-dicarbonyl)bis(azanediyl))(2R,2′R)-bis(3-hydroxypropanoate).

### Example 1. Catalytic Hydrogenation

216 mL of ethyl acetate, 216 mL of ethanol, 9 g of MP-3269, and 1.8 g of a 15 wt % Pd/C catalyst (Evonik) were added to a pressure vessel. The pressure vessel was pressurized with N₂ with agitation (200 RPM) to 15 PSIG and the pressure was released. This was repeated a total of three times. The pressure vessel was then pressurized with H₂ with agitation (200 RPM) to 15 PSIG and the pressure was released. This was repeated a total of two times. The pressure vessel was then again pressurized with H₂ with agitation (200 RPM) to 15 PSIG.

A catalytic hydrogenation reaction took place in the pressure vessel under agitation for 30 minutes while maintaining 15-20 PSIG of H₂. During the reaction, the pressure continuously dropped over three hours and the reaction temperature increased from 16° C. to 22° C. After three hours of reaction, there was a 97% yield of MB-102.

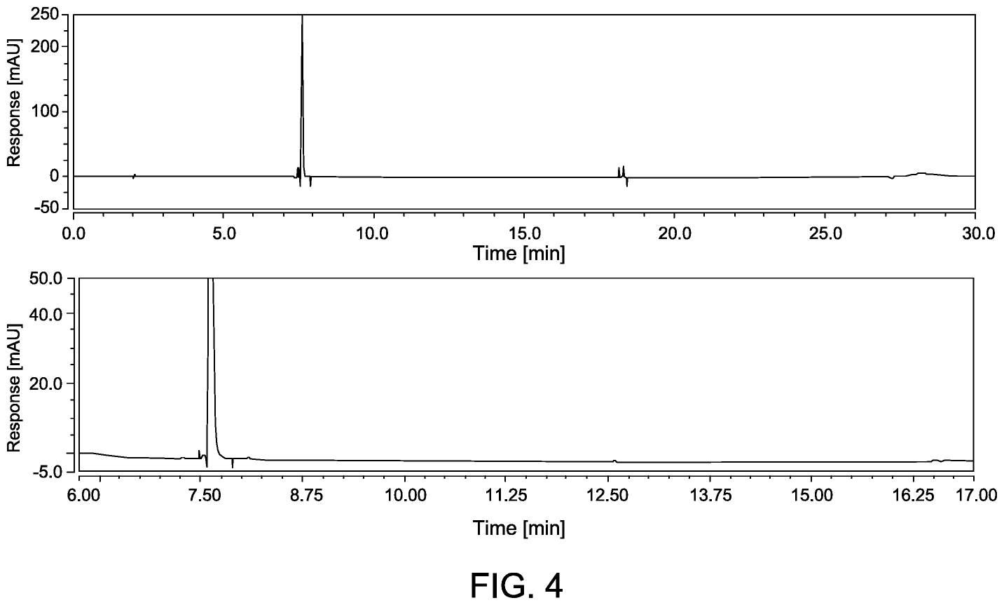

FIG. 4 depicts HPLC chromatograms obtained after the three hours of reaction. The top and bottom chromatograms differ only in scale. The chromatograms were obtained at an absorption wavelength of 264 nm. As can be seen, there is a single, strong MB-102 peak indicating a high purity product.

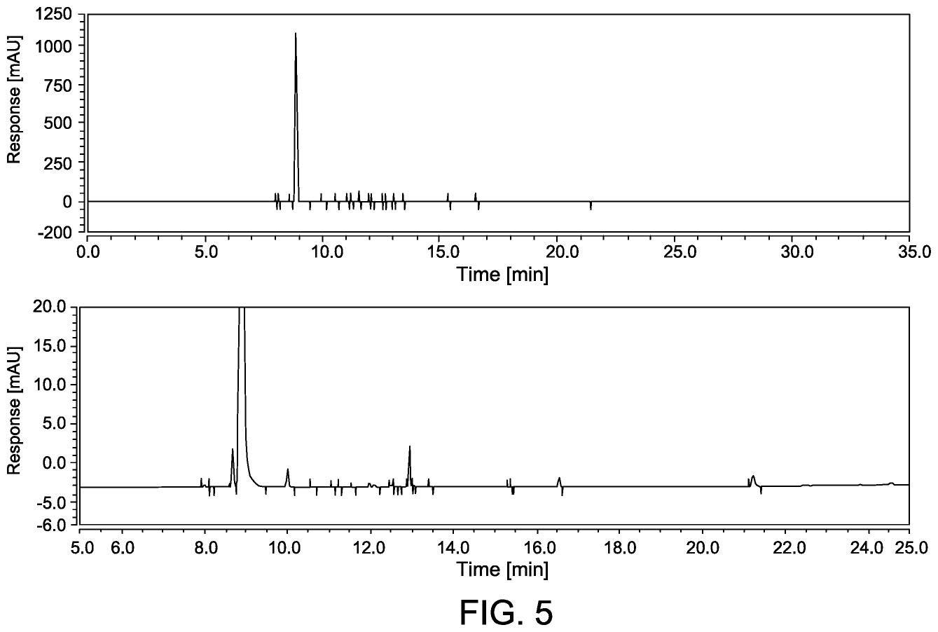

There was no change in yield with an additional two hours of reaction. FIG. 5 depicts a first set of HPLC chromatograms obtained after the additional two hours of reaction. The top and bottom chromatograms differ only in scale. The chromatograms were obtained at an absorption wavelength of 264 nm. As can be seen, there is still a strong MB-102 peak indicating a high purity product. There are also minor amounts of unreacted reactant MP-3269 and impurity MB-102 int. The unreacted reactant and impurity are only visible with a very focused response scale.

The completed reaction product was filtered. A Pd scavenger (SilaMetS) was agitated in the reaction mixture for at least one hour. The reaction was then filtered and left overnight. After one night, the reaction solution was concentrated to 75 mL and then agitated for three hours. The solids in the reaction mixture were perceived as orange and subsequently turned red. The solids were filtered and then washed twice with ethyl acetate. The solids were then dried in a vacuum oven at 55° C. for 48 hours to give a red solid. The final yield of MB-102 was 5.59 g, which is a yield of 92%.

### Example 2. Catalytic Hydrogenation

125 g of MP-3269 and 25 g of 10% Pd/C Evonix Noblyst (50% wet) catalyst were charged to a pressure vessel. The pressure vessel was sealed and evacuated with vacuum to −5 PSIG. 3 L of ethyl acetate were added, followed by 3 L of ethanol. The vessel was placed under a nitrogen atmosphere by pressurizing the vessel to 10 PSIG with N₂ and then releasing the pressure until 1 PSIG was achieved. This was repeated for two additional cycles.

Agitation was started. The vessel was placed under a hydrogen atmosphere by pressurizing the vessel to 10 PSIG with H₂ then releasing the pressure until 1 PSIG was achieved. This was repeated once more. The vessel was then pressurized to 20 PSIG with H₂. The reaction was held under pressure and refilled with H₂ periodically until the pressure stopped decreasing (about 10 hours at this 125 g scale). The purity was evaluated at this point, and it was determined that MP-3269 and MB-102 int were each present in an amount of less than 1 wt %.

The H₂ atmosphere was vented, and the vessel was swapped to a nitrogen atmosphere. N₂ pressure was used to push the slurry out of the pressure vessel into a clean flask kept under N₂ atmosphere. The slurry was filtered through Whatman No 1 filter paper under N₂ atmosphere. 38.75 g of SiliaMetS Thiourea Pd scavenger was charged to the filtrate, the slurry was agitated for 1 hour minimum and then filtered. The solution was concentrated and held for 1 hour minimum. The slurry was then filtered and the solids were washed with ethyl acetate. The solids were dried in a vacuum oven (17 in Hg, 55° C.) to give a red solid. The final yield of MB-102 was 76.7 g, which is a yield of 91%. The purity was over 99% (99.2%).

### Comparative Example 1. Comparative Catalytic Hydrogenation

For a comparative reaction process, the process of Example 1 was followed, except the ethyl acetate was replaced with water. Yields ranged from 35-70%.

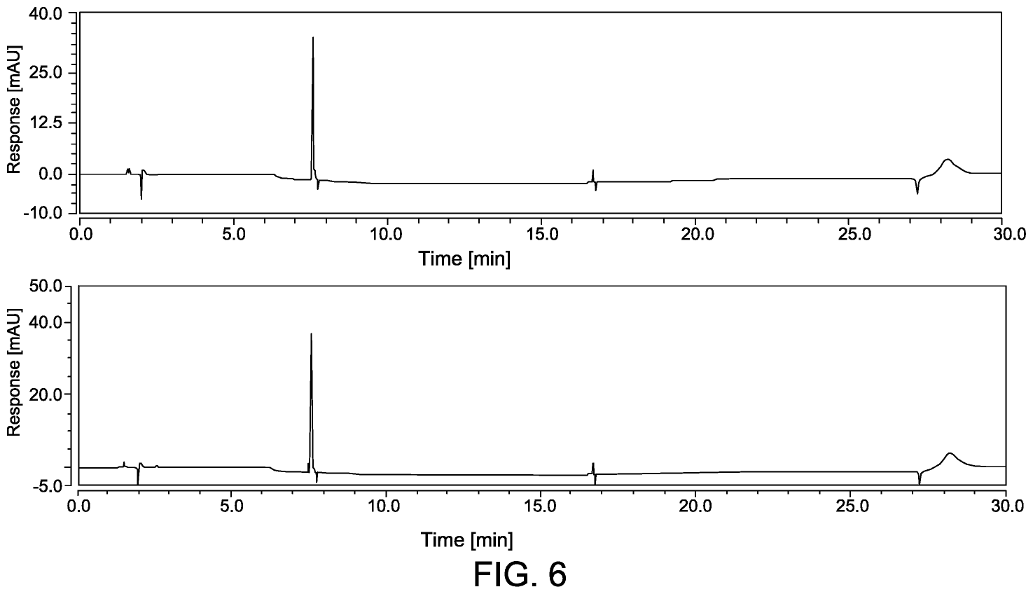

FIG. 6 depicts HPLC chromatograms obtained after completion of the comparative aqueous reaction. The top and bottom chromatograms differ only in scale. The chromatograms were obtained at an absorption wavelength of 264 nm. As can be seen, there is a strong MB-102 peak, as well as an MP-3269 peak that is visible on the same scale, indicating a relatively low purity product.

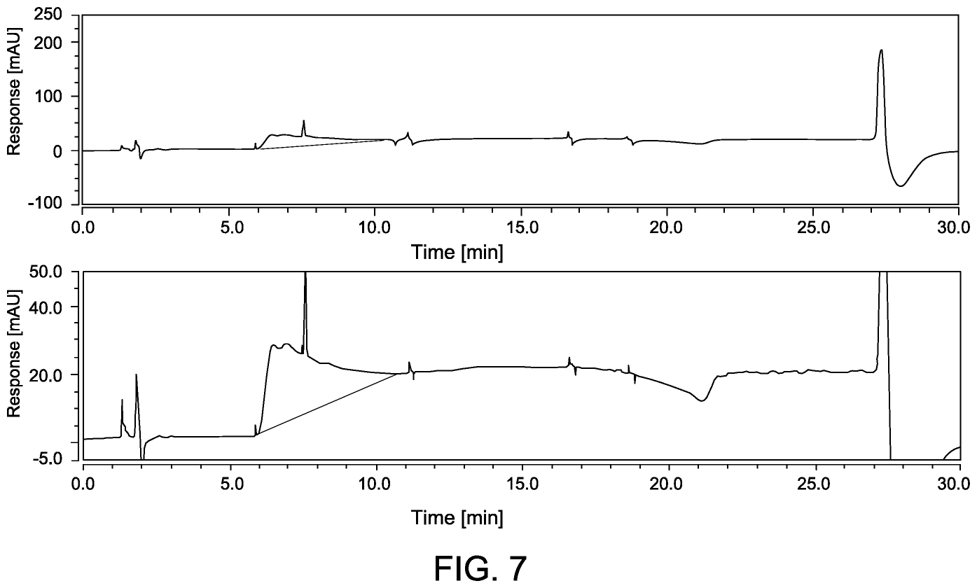

FIG. 7 depicts HPLC chromatograms obtained after completion of the comparative aqueous reaction. The top and bottom chromatograms differ only in scale. The chromatograms were obtained at an absorption wavelength of 215 nm. As can be seen, at this wavelength, there is still a strong MB-102 peak, as well as an MP-3269 peak that is visible on the same scale. There is also a peak at 11.171 corresponding to MB-102 int. These chromatograms indicate a relatively low purity product.

This comparative example demonstrates the detriments of an aqueous reaction system.

As demonstrated herein, catalytic hydrogenation of protected substituted pyrazines in the presence of an organic solvent surprisingly achieves significantly enhanced yields of over 90% compared to catalytic hydrogenation of protected substituted pyrazines in the presence of an aqueous solvent. The benefits achieved in this discovery are particularly useful in the preparation of substituted pyrazines.

### Example 3. Purification

To a round bottom flask was charged DMSO (3.56 kg) followed by crude MB-102 (0.91 kg). DMSO (0.46 kg) was used to rinse the sides of the flask. The slurry was heated to 35-45° C. until the solution was homogenous. The homogenous solution was held at 35-45° C. for at least 30 minutes. IPAc (6.36 kg) was charged at a rate to keep the reaction solution temperature at 35-45° C. The slurry was held for 1-2 hours at 35-45° C. then slowly cooled to 15-25° C. The slurry was held at 15-25° C. for a minimum of 8 hours. IPAc (3.19 kg) was added over at least 1 hour while maintaining a solution temperature of 15-25° C. The slurry was held at 15-25° C. for at least 4 hours. Supernatant concentration was ≤20 mg/mL, with a measured concentration of 13 mg/mL. The slurry was filtered and the solids were washed with IPAc (4.45 kg). The solids were dried under vacuum at 50±5° C. for a minimum of 18 hours to give 1.26 kg of a red solid. A sample was submitted for purity (≥98.0% AUC), impurities (record ≥0.05%), and LoD (record, 36.4%). DMSO (2.13 kg) was charged to a clean vessel. MB-102 (1.26 kg) was charged. The vessel was washed with DMSO (0.40 kg). The slurry was heated to 35-45° C. and held until homogenous. The homogenous solution was held at 35-45° C. for at least 30 minutes. A second clean vessel was charged with water (11.49 kg). The MB-102/DMSO solution was added to the water vessel over at least 1 hour while maintaining a solution temperature of 15-25° C. The slurry was held for at least 4 hours at 15-25° C. The slurry was filtered and washed with water (2×3.62 kg). The solids were dried under vacuum at 50±5° C. for at least 4 hours to give 660 g (73% yield) of MB-102.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where an invention or a portion thereof is defined with an open-ended term such as “comprising,” it should be readily understood that (unless otherwise stated) the description should be interpreted to also describe such an invention using the terms “consisting essentially of” or “consisting of.”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As used herein, the term “about” means plus or minus 10% of the value.

The term “salt thereof” refers to a suitable salt of the compound that this term proceeds. Suitable salts include, without limitation, alkali metal salts, sodium salts, potassium salts, lithium salts, cesuim salts, alkaline metal salts, calcium salts, magnesium salts, halogen salts, chloride salts, bromide salts, iodide salts, sulfate salts, disulfate salts, nitrate salts, phosphate salts, dihydric phosphate salts, hydrophosphate salts, carbonate salts, bicarbonate salts, mesylate salts, and combinations thereof. It is understood that certain compounds of this invention can exist as pure compounds, compounds per se, salts thereof, and combinations thereof.

The term “halogen” includes, without limitation, fluorine, chlorine, bromine and iodine.

The term “amine” includes, without limitation, a functional group comprising a nitrogen atom with a lone pair of electrons. Amines include primary, secondary and tertiary amines.

The term “nitroso” includes, without limitation, a functional group comprising a nitroso group (—N═O).

The term “nitro” includes, without limitation, a functional group comprising a nitro group (—N(═O)—O—).

The term “amide” includes, without limitation, a functional group comprising an amide group (—C(═O)—N—).

The term “ester” includes, without limitation, a functional group comprising an ester group (C(═O)—O—).

The term “carboxyl” includes, without limitation, a functional group comprising a carboxyl group (—C(═O)—O).

What is claimed is:

1. A method of preparing a compound of Formula II or a salt thereof, 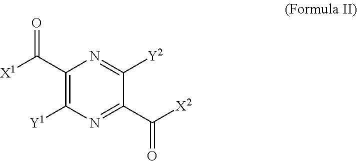
the method comprising:
forming a mixture comprising
a compound of Formula I or a salt thereof, 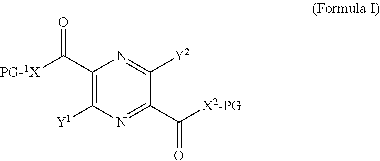
wherein:
PG is a protecting group;
each of Y¹ and Y² is independently selected from the group consisting of hydrogen, halogen, amine, nitroso, nitro, amide, ester, and carboxyl; and
each of X¹ and X² is independently selected from the group consisting of
(i) one or more natural or unnatural α-amino acids or a polypeptide chain that includes one or more natural or unnatural α-amino acids linked together by peptide bonds, or
(ii) N(R¹)(R²) wherein:
each R¹ is independently —(CH₂)ₐ(CH₂CH₂O)_{b}(CH₂)_{c}NR¹⁰CONR¹¹(CH₂)_{d}(CH₂CH₂O)ₑR²⁰, —(CH₂)ₐ(CH₂CH₂O)_{b}(CH2)_{c}NR¹²CSNR¹³(CH₂)_{d}(CH₂CH₂O)ₑR²¹, —(CH₂)ₐ(CH₂CH₂O)_{b}(CH₂)_{c}CONR¹⁴(CH₂)_{d}(CH₂CH₂O)ₑR²², —(CH₂)ₐ(CH₂CH₂O)_{b}(CH₂)_{c}NR¹⁵SO₂(CH₂)_{d}(CH₂CH₂O)ₑR²³, —(CH₂)ₐ(CH₂CH₂O)_{b}(CH₂)_{c}SO₂NR¹⁶(CH₂)_{d}(CH₂CH₂O)ₑR²⁴, —(CH2)ₐ(CH₂CH₂O)_{b}(CH₂)_{c}NR¹⁷CO(CH₂)_{d}(CH₂CH₂O)ₑR²⁵, —(CH2)ₐ(CH₂CH₂O)_{b}(CH₂)_{c}NR¹⁸CO₂(CH₂)_{d}(CH₂CH₂O)ₑR²⁶, or —(CH₂)ₐ(CH₂CH₂O)_{b}(CH₂)_{c}OC(O)NR¹⁹CO₂(CH₂)_{d}(CH₂CH₂O)ₑR²⁷; —(CH₂)_{c}OR⁶⁸, —CH₂(CHOH)_{c}R⁶⁹, —CH₂(CHOH)_{c}O₂H, —(CHCO₂H)_{c}CO₂H, —(CH₂)_{c}NR⁷⁰R⁷¹, —CH[(CH₂)_{f}NH₂]_{c}CO₂H, —CH[(CH₂)_{f}NH₂]_{c}CH₂OH, —CH₂(CHNH₂)_{c}CH2NR⁷²R⁷³, —(CH₂CH₂O)ₑR⁷⁴, —(CH₂)ₜCO(CH₂CH₂O)ₑR⁷⁵, —(CH₂)ᵤ(CH₂CH₂O)ⱼ(CH₂)ₖNR⁵⁸C(O)NR⁵⁹(CH₂)ₗ(CH₂CH₂O)ₒR⁷⁶, —(CH₂)ᵤ(CH₂CH₂O)ⱼ(CH₂)ₖNR⁶⁰C(S)NR⁶¹(CH₂)ₗ(CH₂CH₂O)ₒR⁷⁷, —(CH₂)ᵤ(CH₂CH₂O)ⱼ(CH₂)ₖC(O)NR⁶²(CH₂)ₗ(CH₂CH₂O)ₒR⁷⁸, —(CH₂)ᵤ(CH₂CH₂O)ⱼ(CH₂)ₖS(O)₂NR⁶³(CH₂)ₗ(CH₂CH₂O)ₒR⁷⁹, —(CH₂)ᵤ(CH₂CH₂O)ⱼ(CH₂)ₖNR⁶⁴S(O)₂(CH₂)ₗ(CH₂CH₂O)ₒR⁸⁰, —(CH₂)ᵤ(CH₂CH₂O)ⱼ(CH₂)ₖNR⁶⁵C(O)(CH₂)ₗ(CH₂CH₂O)ₒR⁸¹, —(CH₂)ᵤ(CH₂CH₂O)ⱼ(CH₂)ₖNR⁶⁶C(O)O(CH₂)ₗ(CH₂CH₂O)ₒR⁸², or —(CH₂)ᵤ(CH₂CH₂O)ⱼ(CH₂)ₖOC(O)NR⁶⁷(CH₂)ₗ(CH₂CH₂O)ₒR⁸³, —(CH₂)ₐSO₃H, —(CH₂)ₐSO₃⁻, —(CH₂)ₐOSO₃H, —(CH₂)ₐOSO₃⁻, —(CH₂)ₐNHSO₃H, —(CH₂)ₐNHSO₃⁻, —(CH₂)ₐPO₃H₂, —(CH₂)ₐPO₃H⁻, —(CH₂)ₐPO₃²⁻, —(CH₂)ₐOPO₃H₂, —(CH₂)ₐOPO₃H⁻, or —(CH₂)ₐOPO³;
each of R², R¹⁰, R¹¹, R¹², R¹³, R¹⁴, R¹⁵, R¹⁶, R¹⁷, R¹⁸, R¹⁹, R⁵⁸, R⁵⁹, R⁶⁰, R⁶¹, R⁶², R⁶³, R⁶⁴, R⁶⁵, R⁶⁶ and R⁶⁷ is independently —H or —CH₃;
each of R²⁰, R²¹, R²², R²³, R²⁴, R²⁵, R²⁶, and R²⁷ is independently —H, —CH₃, —(CH₂)_{f}NR²⁸C(O)NR²⁹(CH₂)_{g}(CH₂CH₂O)ₕR³⁸, —(CH₂)_{f}NR³⁰CSNR³¹(CH₂)_{g}(CH₂CH₂O)ₕR³⁹, —(CH₂)_{f}C(O)NR³²(CH₂)_{g}(CH₂CH₂O)ₕR⁴⁰, —(CH₂)_{f}S(O)₂NR³³(CH₂)_{g}(CH₂CH₂O)ₕR⁴¹, —(CH₂)_{f}NR³⁴S(O)₂(CH₂)_{g}(CH₂CH₂O)ₕR⁴², —(CH₂)_{f}NR³⁵C(O)(CH₂)_{g}(CH₂CH₂O)ₕR⁴³, —(CH₂)_{f}NR³⁶C(O)O(CH₂)_{g}(CH₂CH₂O)ₕR⁴⁴, —(CH₂)_{f}OC(O)NR³⁷(CH₂)_{g}(CH₂CH₂O)ₕR⁴⁵, —CO(AA), or —CONH(PS);
each of R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷ and R⁴⁸ is independently —H or —CH₃;
each of R⁶⁸, R⁶⁹, R⁷⁰, R⁷¹, R⁷², R⁷³, R⁷⁴, R⁷⁵, R⁷⁶, R⁷⁷, R⁷⁸, R⁷⁹, and R⁸⁰ is independently —H —CH₃, —(CH₂)ₚS(O)₂NR⁸⁴(CH₂)_{q}(CH₂CH₂O)ₛR⁸¹, —(CH₂)ₚNR⁸⁵S(O)₂(CH₂)_{q}(CH₂CH₂O)ₛR⁸³, —(CH₂)ₚNR⁸⁶C(O)(CH₂)_{q}(CH₂CH₂O)ₛR⁸⁵, —(CH₂)ₚNR⁸⁶C(O)O(CH₂)_{q}(CH₂CH₂O)ₛR⁸⁷, or —(CH₂)ₚOC(O)NR⁸⁸(CH₂)_{q}(CH₂CH₂O)ₛR⁸⁹;
each of R⁸¹, R⁸², R⁸³, R⁸⁴, R⁸⁵, R⁸⁶, R⁸⁷, R⁸⁸, and R⁸⁹ is independently —H or —CH₃;
each (AA) is independently one or more natural or unnatural α-amino acids or a polypeptide chain that includes one or more natural or unnatural α-amino acids linked together by peptide bonds;
each (PS) is independently a sulfated or non-sulfated polysaccharide chain comprising one or more monosaccharide units connected by glycosidic linkages;
each of t and u is independently 1, 2, 3, 4, or 5;
each of a, d, g, l, and q is independently 0, 1, 2, 3, 4, 5, 6, 7, 8, 9 or 10;
each of c, f, k, and p is independently 0, 1, 2, 3, 4, 5, 6, 7, 8, 9 or 10; and
each of b, j, e, h, o, and s is independently 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100;
an organic solvent;
a catalyst; and
a hydrogenation agent; and
reacting the mixture.

2. The method of claim 1, wherein each of X¹ and X² is D-serine, each of Y¹ and Y² is amine, PG is a benzyl protecting group, and the compound of Formula I is dibenzyl 2,2′-((3,6-diaminopyrazine-2,5-dicarbonyl)bis(azanediyl))(2R,2′R)-bis(3-hydroxypropanoate) of the following structure:

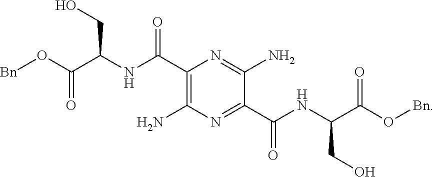 [SMILES: Nc1nc(C(=O)N[C@H](CO)C(=O)OCc2ccccc2)c(N)nc1C(=O)N[C@H](CO)C(=O)OCc1ccccc1]

3. The method of claim 1, wherein each of X¹ and X² is D-serine, each of Y¹ and Y² is amine, and the compound of Formula II is (2R,2′R)-2,2′-((3,6-diaminopyrazine-2,5-dicarbonyl)bis(azanediyl))bis(3-hydroxypropanoic acid) of the following structure:

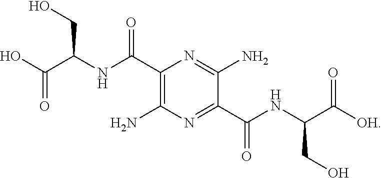 [SMILES: Nc1nc(C(=O)N[C@H](CO)C(=O)O)c(N)nc1C(=O)N[C@H](CO)C(=O)O]

4. The method of claim 1, wherein PG is a protecting group selected from the group consisting of benzyl (“Bn”), t-butyl, p-t-butylbenzyl, methyl, p-methoxybenzyl, p-sec-butyl, p-i-propylbenzyl, p-n-propylbenzyl, p-ethylbenzyl, and p-methylbenzyl.

5. The method of claim 1, wherein the organic solvent comprises a solvent selected from the group consisting of alcohols, methanol, ethanol, propanol, isopropanol, butanol, sec-butanol, t-butanol, acetates, methyl acetate, ethyl acetate, acetic acid, n-propionic acid, n-butanoic acid, iso-butyric acid, and combinations thereof.

6. The method of claim 1, wherein the hydrogenation agent comprises a hydrogenation agent selected from the group consisting of hydrogenation hydrogen sources, hydrogen (H₂), formic acid, formate, isopropanol, dihydroanthracene, 1,4-cyclohexadiene, 1,3-cyclohexadiene, 1-methyl-1,4-cyclohexadiene, and combinations thereof.

7. The method of claim 1, wherein the catalyst comprises a catalyst selected from the group consisting of hydrogenation catalysts, noble metal catalysts, palladium (Pd), platinum (Pt), gold (Au), rhodium (Rh), ruthenium (Ru), silver (Ag), osmium (Os), iridium (Ir), transition metal catalysts, nickel (Ni), Raney nickel, Urushibara nickel, iron (Fe), molybdenum (Mo), cobalt (Co), copper (Cu), chromium (Cr), catalysts supported on a catalyst support, catalysts supported on carbon, catalysts supported on alumina, catalysts supported on silica, Pd/C, Pt/C, Pd/C containing water in an amount in a range of from about 1% to about 50%, Pd/C containing about 50% water, and combinations thereof.

8. The method of claim 1, wherein the method step of reacting the mixture occurs at a reaction temperature in the range of about −20° C. to about 100° C.

9. The method of claim 1, wherein the method step of reacting the mixture occurs at atmospheric pressure.

10. The method of claim 1, wherein the method step of reacting the mixture occurs at an elevated reaction pressure.

11. The method of claim 1, wherein the method step of reacting the mixture occurs at a reaction pressure in the range of about 1 PSIG to about 50 PSIG.

12. The method of claim 1, wherein the method step of reacting the mixture occurs during a reaction time in the range of about 1 hour to about 48 hours.

13. The method of claim 1, wherein the mixture is substantially free of aqueous solvents.

14. The method of claim 1, wherein the mixture further comprises an aqueous solvent.

15. The method of claim 1, further comprising a method step selected from the group consisting of filtering, centrifuging, centrifugal filtration, isolating, washing, drying, concentrating, catalyst scavenging, purifying, and combinations thereof.

16. The method of claim 1, further comprising:
purifying the compound of Formula II or a salt thereof with a first solvent and an anti-solvent; and
optionally removing the first solvent from the compound of Formula II or a salt thereof.

17. The method of claim 16, wherein:
the first solvent is selected from the group consisting of organic solvents, dimethyl sulfoxide (DMSO), and combinations thereof; and
the anti-solvent is selected from the group consisting of acetates, ethyl acetate, isopropyl acetate, and combinations thereof.

18. A method of purifying a compound of Formula II or a salt thereof, 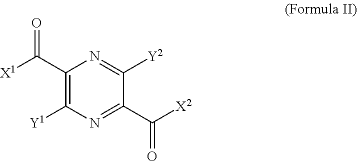
the method comprising:
precipitating the compound of Formula II or a salt thereof with a first solvent and an anti-solvent; and
optionally removing the first solvent from the compound of Formula II or a salt thereof;
wherein:
each of Y¹ and Y² is independently selected from the group consisting of hydrogen, halogen, amine, nitroso, nitro, amide, ester, and carboxyl; and
each of X¹ and X² is independently selected from the group consisting of
(i) one or more natural or unnatural α-amino acids or a polypeptide chain that includes one or more natural or unnatural α-amino acids linked together by peptide bonds, or
(ii) N(R¹)(R²) wherein:
each R¹ is independently —(CH₂)ₐ(CH₂CH₂O)_{b}(CH₂)_{c}NR¹⁰CONR¹¹(CH₂)_{d}(CH₂CH₂O)ₑR²⁰, —(CH₂)ₐ(CH₂CH₂O)_{b}(CH₂)_{c}NR¹²CSNR¹³(CH₂)_{d}(CH₂CH₂O)ₑR²¹, —(CH₂)ₐ(CH₂CH₂O)_{b}(CH₂)_{c}CONR¹⁴(CH₂)_{d}(CH₂CH₂O)ₑR²², —(CH₂)ₐ(CH₂CH₂O)_{b}(CH₂)_{c}NR¹⁵SO₂(CH₂)_{d}(CH₂CH₂O)ₑR²³, —(CH₂)ₐ(CH₂CH₂O)_{b}(CH₂)_{c}SO₂NR¹⁶(CH₂)_{d}(CH₂CH₂O)ₑR²⁴, —(CH2)ₐ(CH₂CH₂O)_{b}(CH₂)_{c}NR¹⁷CO(CH₂)_{d}(CH₂CH₂O)ₑR²⁵, —(CH2)ₐ(CH₂CH₂O)_{b}(CH₂)_{c}NR¹⁸CO₂(CH₂)_{d}(CH₂CH₂O)ₑR²⁶, or —(CH₂)ₐ(CH₂CH₂O)_{b}(CH₂)_{c}OC(O)NR¹⁹CO₂(CH₂)_{d}(CH₂CH₂O)ₑR²⁷; —(CH₂)_{c}OR⁶⁸, —CH₂(CHOH)_{c}R⁶⁹, —CH₂(CHOH)_{c}CO₂H, —(CHCO₂H)_{c}CO₂H, —(CH₂)_{c}NR⁷⁰R⁷¹, —CH[(CH₂)_{f}NH₂]_{c}CO₂H, —CH[(CH₂)_{f}NH₂]_{c}CH₂OH, —CH₂(CHNH₂)_{c}CH2NR⁷²R⁷³, —(CH₂CH₂O)ₑR⁷⁴, —(CH₂)ₜCO(CH₂CH₂O)ₑR⁷⁵, —(CH₂)ᵤ(CH₂CH₂O)ⱼ(CH₂)ₖNR⁵⁸C(O)NR⁵⁹(CH₂)ₗ(CH₂CH₂O)ₒR⁷⁶, —(CH₂)ᵤ(CH₂CH₂O)ⱼ(CH₂)ₖNR⁶⁰C(S)NR⁶¹(CH₂)ₗ(CH₂CH₂O)ₒR⁷⁷, —(CH₂)ᵤ(CH₂CH₂O)ⱼ(CH₂)ₖC(O)NR⁶²(CH₂)ₗ(CH₂CH₂O)ₒR⁷⁸, —(CH₂)ᵤ(CH₂CH₂O)ⱼ(CH₂)ₖS(O)₂NR⁶³(CH₂)ₗ(CH₂CH₂O)ₒR⁷⁹, —(CH₂)ᵤ(CH₂CH₂O)ⱼ(CH₂)ₖNR⁶⁴S(O)₂(CH₂)ₗ(CH₂CH₂O)ₒR⁸⁰, —(CH₂)ᵤ(CH₂CH₂O)ⱼ(CH₂)ₖNR⁶⁵C(O)(CH₂)ₗ(CH₂CH₂O)ₒR⁸¹, —(CH₂)ᵤ(CH₂CH₂O)ⱼ(CH₂)ₖNR⁶⁶C(O)O(CH₂)ₗ(CH₂CH₂O)ₒR⁸², or —(CH₂)ᵤ(CH₂CH₂O)ⱼ(CH₂)ₖOC(O)NR⁶⁷(CH₂)ₗ(CH₂CH₂O)ₒR⁸³, —(CH₂)ₐSO₃H, —(CH₂)ₐSO₃⁻, —(CH₂)ₐOSO₃H, —(CH₂)ₐOSO₃⁻, —(CH₂)ₐNHSO₃H, —(CH₂)ₐNHSO₃⁻, —(CH₂)ₐPO₃H₂, —(CH₂)ₐPO₃H⁻, —(CH₂)ₐPO₃²⁻, —(CH₂)ₐOPO₃H₂, —(CH₂)ₐOPO₃H⁻, or —(CH₂)ₐOPO³;
each of R², R¹⁰, R¹¹, R¹², R¹³, R¹⁴, R¹⁵, R¹⁶, R¹⁷, R¹⁸, R¹⁹, R⁵⁸, R⁵⁹, R⁶⁰, R⁶¹, R⁶², R⁶³, R⁶⁴, R⁶⁵, R⁶⁶ and R⁶⁷ is independently —H or —CH₃;
each of R²⁰, R²¹, R²², R²³, R²⁴, R²⁵, R²⁶, and R²⁷ is independently —H, —CH₃, —(CH₂)_{f}NR²⁸C(O)NR²⁹(CH₂)_{g}(CH₂CH₂O)ₕR³⁸, —(CH₂)_{f}NR³⁰CSNR³¹(CH₂)_{g}(CH₂CH₂O)ₕR³⁹, —(CH₂)_{f}C(O)NR³²(CH₂)_{g}(CH₂CH₂O)ₕR⁴⁰, —(CH₂)_{f}S(O)₂NR³³(CH₂)_{g}(CH₂CH₂O)ₕR⁴¹, —(CH₂)_{f}NR³⁴S(O)₂(CH₂)_{g}(CH₂CH₂O)ₕR⁴², —(CH₂)_{f}NR³⁵C(O)(CH₂)_{g}(CH₂CH₂O)ₕR⁴³, —(CH₂)_{f}NR³⁶C(O)O(CH₂)_{g}(CH₂CH₂O)ₕR⁴⁴, —(CH₂)_{f}OC(O)NR³⁷(CH₂)_{g}(CH₂CH₂O)ₕR⁴⁵, —CO(AA), or —CONH(PS);
each of R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷ and R⁴⁸ is independently —H or —CH₃;
each of R⁶⁸, R⁶⁹, R⁷⁰, R⁷¹, R⁷², R⁷³, R⁷⁴, R⁷⁵, R⁷⁶, R⁷⁷, R⁷⁸, R⁷⁹, and R⁸⁰ is independently —H, —CH₃, —(CH₂)ₚS(O)₂NR⁸⁴(CH₂)_{q}(CH₂CH₂O)ₛR⁸¹, —(CH₂)ₚNR⁸⁵S(O)₂(CH₂)_{q}(CH₂CH₂O)ₛR⁸³, —(CH₂)ₚNR⁸⁶C(O)(CH₂)_{q}(CH₂CH₂O)ₛR⁸⁵, —(CH₂)ₚNR⁸⁶C(O)O(CH₂)_{q}(CH₂CH₂O)ₛR⁸⁷, or —(CH₂)ₚOC(O)NR⁸⁸(CH₂)_{q}(CH₂CH₂O)ₛR⁸⁹;
each of R⁸¹, R⁸², R⁸³, R⁸⁴, R⁸⁵, R⁸⁶, R⁸⁷, R⁸⁸, and R⁸⁹ is independently —H or —CH₃;
each (AA) is independently one or more natural or unnatural α-amino acids or a polypeptide chain that includes one or more natural or unnatural α-amino acids linked together by peptide bonds;
each (PS) is independently a sulfated or non-sulfated polysaccharide chain comprising one or more monosaccharide units connected by glycosidic linkages;
each of t and u is independently 1, 2, 3, 4, or 5;
each of a, d, g, l, and q is independently 0, 1, 2, 3, 4, 5, 6, 7, 8, 9 or 10;
each of c, f, k, and p is independently 0, 1, 2, 3, 4, 5, 6, 7, 8, 9 or 10; and
each of b, j, e, h, o, and s is independently 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100;

19. The method of claim 18, wherein removing the first solvent from the compound of Formula II or a salt thereof comprises removing the first solvent from the compound of Formula II or a salt thereof with a solvent precipitation process comprising:
washing the compound of Formula II or a salt thereof with the first solvent; and
precipitating the compound of Formula II or a salt thereof by mixing the washed compound of Formula II or a salt thereof with a second solvent.

20. The method of claim 19, wherein:
the first solvent is selected from the group consisting of organic solvents, dimethyl sulfoxide (DMSO), and combinations thereof; and
the second solvent is selected from the group consisting of aqueous solvents, water, and combinations thereof.
